(19) United States
(12) Patent Application Publication
(10) Pub. No.: US 2014/0080132 A1
(43) Pub. Date: Mar. 20, 2014

(54) METHOD AND KIT FOR PREDICTING CYTOTOXICITY

(71) Applicants: Hitachi Chemical Company, Ltd., Tokyo (JP); National Cancer Center, Tokyo (JP); Hitachi Chemical Research Center, Inc., Irvine, CA (US)

(72) Inventors: Kazuhiko Obara, Hitachi (JP); Hiroshi Izutsu, Tokyo (JP); Masato Mitsuhashi, Irvine, CA (US); Fumiaki Koizumi, Tokyo (JP); Kenji Tamura, Tokyo (JP); Mayu Yunokawa, Tokyo (JP); Yasuo Kodera, Tokyo (JP); Fumiko Taguchi, Tokyo (JP)

(21) Appl. No.: 14/028,221

(22) Filed: Sep. 16, 2013

Related U.S. Application Data
(60) Provisional application No. 61/702,147, filed on Sep. 17, 2012.

(51) Int. Cl. C12Q 1/68

(52) U.S. Cl. CPC C12Q 1/6886
USPC 435/6.12; 536/24.33; 536/24.31

(57) ABSTRACT

A method for predicting ADCC activity in a subject, the method comprising the steps of: (a) preparing a biological sample from the subject, said sample including a leukocyte, (b) bringing a portion of the biological sample and an antibody into contact with each other, (c) detecting expression of at least one marker of ADCC activity selected from the group consisting of tumor necrosis factor super family 15, chemokine CXCL3, and interleukin 6 in the leukocyte in (i) the portion of the sample brought into contact with the antibody and in (ii) another portion of the sample not brought into contact with the antibody, (d) comparing an expression level in portion (i) with the expression level in portion (ii); and (e) predicting presence of the cytotoxic activity when the expression level in portion (i) is higher than the expression level in portion (ii)

### CROSS-REFERENCE TO RELATED APPLICATION

This application claims priority to Provisional Application No. 61/702,147, filed on Sep. 17, 2012, which is hereby incorporated by reference in its entirety.

### BACKGROUND OF THE INVENTION

1. Field of the Invention

The present invention relates to a method and a kit for predicting a cytotoxic activity, in particular, an antibody-dependent cellular cytotoxic (ADCC) activity. Specifically, it relates to a method and a kit for predicting a cytotoxic activity by detecting expression of a specific gene. The present invention relates to a method for evaluating drug responsiveness of a patient, and a method for screening for an antibody having a cytotoxic activity.

2. Related Background Art

An antibody-dependent cellular cytotoxic (ADCC) activity is a cytotoxic activity induced in an antibody-dependent manner in which an antibody bound to a target cell is bound to an Fc receptor on an effector cell such as a natural-killer cell (NK cell) and macrophage (Mitsuo, S. et al., Trends in Glycoscience and Glycotechnology 18: 129-136, 2006).

Regarding the ADCC activity, it is reported that the ADCC activity is one of the antitumor mechanisms of anticancer antibody drugs such as rituximab (trade name: Rituxan (registered trademark)), trastuzumab (trade name: Herceptin (registered trademark)), and cetuximab (trade name: Erbitux (registered trademark)) (Mitsuo, S. et al., Trends in Glycoscience and Glycotechnology 18: 129-136, 2006; Reff, M. E. et al., Blood 83: 435-445, 1994; Kurai, J. et al., Clin. Cancer Res. 13: 1552-1561, 2007; Clynes, R. A. et al., Nature Medicine 6: 443-446, 2000). Therefore, for expecting higher clinical effects, antibody drugs exhibiting a high ADCC activity has been attempted to be developed (Mitsuo, S. et al., Trends in Glycoscience and Glycotechnology 18: 129-136, 2006).

The ADCC activity in an individual is known to correlate with the response of the individual to the antibody drug. For example, an effect of treatment by trastuzumab as an anti-HER2 humanized antibody on a breast cancer patient correlates to the ADCC activity of the patient (Gennari, R. et al., Clin. Cancer Res. 10: 5650-5655, 2004; Beano, A. et al., Journal of Translational Medicine 6: 25, 2008). Therefore, by measuring the ADCC activity of a patient before using the antibody drug, the effect of the antibody drug can be predicted.

As mentioned above, the concept of the ADCC activity has been established, but a physiological role and a pathological role of the ADCC activity in clinical applications have not been understood, and what genes are involved in the mechanism of the ADCC activity has not been identified specifically.

Measurement of the ADCC activity has been carried out in various methods. For example, the measurement is carried out by measuring release of radioisotope (chromium or the like) from a target cell in the presence of an antibody and an effector cell (Reff, M. E. et al., Blood 83: 435-445, 1994; Kurai, J. et al., Clin. Cancer Res. 13: 1552-1561, 2007; Clynes, R. A. et al., Nature Medicine 6: 443-446, 2000; Gennari, R. et al., Clin. Cancer Res. 10: 5650-5655, 2004; Beano, A. et al., Journal of Translational Medicine 6: 25, 2008) or by measuring release of fluorescent dye (calcein or the like). However, problems of the conventional methods are, for example, that it takes a long time to carry out measurement because it is necessary to culture living cells, and that measurement results vary depending upon culture conditions.

On the other hand, it is reported that examples of the genes expressed in response to stimulation by an antibody include a tumor necrosis factor (TNF) super family and chemokines (WO 06/133399). WO 06/133399 discloses that expression of genes of the subgroups 2, 8, 14, 15 and 18 of the TNF super family, as well as genes of chemokines CCL-3, CCL-20, CXCL-1, CXCL-2, CXCL-3, IL-8 and IL-1B are induced, in response to the stimulation to the target cell by heat-aggregated human IgG (heat-aggregated human IgG, HAG) antibody. This document discloses that prognosis of tumor can be predicted from the change in the expression level of the TNF super family genes or chemokine genes. Besides, as to the immune response to inflammatory bowel diseases (for example, Crohn's disease) and response to drugs, as genes expressed by response to the stimulation to the target cell by an antibody, genes encoding the TNF super family (TNFSF), chemokine and interleukin are reported (Mitsuhashi, M. and Targan, S. R., Inflamm Bowel Dis. 14: 1061-1067, 2008; Mitsuhashi, M. et al., Pharm. Res. 25: 1116-1124, 2008). However, the documents do not describe the relation between such genes and the ADCC activity.

### SUMMARY OF THE INVENTION

### Problems to be Solved by the Invention

An object of the present invention is to provide means and a method for predicting a cytotoxic activity, in particular, an ADCC activity more simply, more rapidly, and with higher degree of accuracy. Another object of the present invention is to provide a method for evaluating responsiveness to drugs having a mechanism of action based on the cytotoxic activity or a method for screening for an antibody having the cytotoxic activity.

### Means to Solve the Problem

The present inventors have keenly investigated to solve the above-mentioned problems, and, as a result, they have found that the change in the expression level in response to antibody stimulation of tumor necrosis factor super family 15 (TNFSF15), chemokine CXCL3 (CXCL3), or interleukin 6 (IL-6) in the leukocyte is correlated with the cytotoxic activity, in particular, the ADCC activity. Such a finding is beyond prediction and surprising because genes related to the ADCC activity have not conventionally been reported. The present inventors have found that the change in the gene expression in response to the antibody stimulation of chemokine CXCL1(CXCL1) and tumor necrosis factor super family 2 (TNFSF2) in addition to the above-mentioned tumor necrosis factor super family 15 (TNFSF15) and chemokine CXCL3 (CXCL3) in the leukocyte in the peripheral blood of a cancer patient is related to results of treatment of the patient with the use of drugs having a mechanism of action based on the cytotoxic activity, and have obtained a finding that the prediction of the cytotoxic activity can be applied for various applications of use.

That is to say, the present invention relates to the following (1) to (4).

(1) A method for predicting a cytotoxic activity, the method comprises the steps of:

(a) preparing a biological sample including a leukocyte;

(b) bringing the biological sample and an antibody into contact with each other;

(c) detecting expression of at least one marker of cytotoxic activity selected from the group consisting of tumor necrosis factor super family 15, chemokine CXCL3, and interleukin 6 in the leukocyte;

(d) comparing an expression level of a case where the sample is brought into contact with the antibody and a case where the sample is not brought into contact with the antibody; and

(e) predicting that cytotoxic activity is present when the expression level in the case where the sample is brought into contact with the antibody is higher than the expression level in the case where the sample is not brought into contact with the antibody.

In the above-mentioned method, the cytotoxic activity may be an antibody-dependent cellular cytotoxic activity.

In the above-mentioned method, examples of antibodies to be used include heat-aggregated IgG and antibody drugs. Such antibody drugs are not limited, but examples thereof include drugs selected from the group consisting of abciximab, adalimumab, alemtuzumab, basiliximab, bevacizumab, cetuximab, daclizumab, eculizumab, efalizumab, gemtuzumab ozogamicin, ibritumomab tiuxetan, infliximab, muromonab, natalizumab, omalizumab, palivizumab, panitumumab, ranibizumab, rituximab, tositumomab, tocilizumab, golimumab, canakinumab, ustekinumab, ofatumumab, denosumab, motavizumab, raxibacumab, belimumab, ipilimumab, brentuximab vedotin, and trastuzumab. In several embodiments, the method further comprises an additional step (f) of administering an antibody to said subject. In several such embodiments, the antibody is not heat aggregated.

In the above-mentioned method, the antibody may be brought into contact with the biological sample with the antibody being thermally denatured or as a complex with an antigen against the antibody.

In the above-mentioned method, detection of expression may be carried out by detecting mRNA.

In the above-mentioned method, expression of the tumor necrosis factor super family 15 may be detected.

(2) A method for evaluating drug responsiveness of a patient to an antibody drug; the method comprises the steps of:

(a) preparing a biological sample including a leukocyte from a patient;

(b) bringing the biological sample and an antibody into contact with each other;

(c) detecting expression of at least one selected marker from the group consisting of tumor necrosis factor super family 15, chemokine CXCL3, chemokine CXCL1, and tumor necrosis factor super family 2 in the leukocyte;

(d) comparing an expression level in a case where the sample is brought into contact with the antibody and an expression level in a case where the sample is not brought into contact with the antibody so as to predict the cytotoxic activity (for example, ADCC activity); and

(e) determining that the drug responsiveness of the patient to the antibody drug is high when the cytotoxic activity of the patient is high.

In the above-mentioned method, examples of the antibodies to be brought into contact with the biological sample may be one or more selected from the group consisting of heat-aggregated IgG, an anticancer drug, an antiviral agent, an antiinflammatory agent, a rejection inhibitor and an antitumor drug.

In the above-mentioned method, examples of the antibody drug include one selected from the group consisting of an anticancer drug, an antiviral agent, an antiinflammatory agent, a rejection inhibitor, and an antitumor drug. A specific antibody drug includes trastuzumab. In several embodiments, the method further comprises administering an antibody to said patient. In several embodiments, the administered antibody is not heat aggregated.

The above-mentioned method may further include a step of detecting expression of HER2 protein antigen in a patient.

(3) A method for screening for an antibody having a cytotoxic activity (for example, ADCC activity), the method comprises the steps of:

(a) bringing the subject antibody and a leukocyte into contact with each other;

(b) detecting expression of at least one marker selected from the group consisting of tumor necrosis factor super family 15, chemokine CXCL3, and interleukin 6 in the leukocyte; and

(c) selecting a subject antibody as an antibody having the cytotoxic activity when an expression level in a case where the leukocyte is brought into contact with the subject antibody is higher than an expression level in a case where the leukocyte is not brought into contact with the subject antibody.

(4) A kit for predicting a cytotoxic activity (for example, ADCC activity) including means for detecting expression of at least one selected from the group consisting of tumor necrosis factor super family 15, chemokine CXCL3, and interleukin 6.

In the above-mentioned kit, as means for detecting expression, for example, a primer and/or probe may be used.

Furthermore, the present invention relates to the following (5).

(5) A method for analyzing data for evaluating drug responsiveness of a patient to an antibody drug, the method comprises the steps of:

(a) obtaining a biological sample including a leukocyte obtained from the patient;

(b) bringing the biological sample and an antibody into contact with each other;

(c) obtaining data of the expression level by detecting expression of at least one marker selected from the group consisting of tumor necrosis factor super family 15, chemokine CXCL3, chemokine CXCL1, and tumor necrosis factor super family 2 in the leukocyte;

(d) comparing data of the expression level in a case where the sample is brought into contact with the antibody and data of the expression level in a case where the sample is not brought into contact with the antibody; and

(e) analyzing data of the expression level in the case where the sample is brought into contact with the antibody based on evaluation criteria that the expression level in the case where the sample is brought into contact with the antibody is higher than the expression level in the case where the sample is not brought into contact with the antibody. Furthermore, the present invention relates to the following (6).

(6) A method for predicting antibody-dependent cellular cytotoxic (ADCC) activity in a subject, the method comprising the steps of:

(a) preparing a biological sample from the subject, said sample including a leukocyte;

(b) bringing a portion of the biological sample and an antibody into contact with each other, wherein the antibody is heat aggregated in the step (b);

(c) detecting expression of at least one marker of ADCC activity selected from the group consisting of tumor necrosis factor super family 15, chemokine CXCL3, and interleukin 6 in the leukocyte in (i) the portion of the sample brought into contact with the antibody and in (ii) another portion of the sample not brought into contact with the antibody by a method comprising:
(i) contacting RNA from each of the samples with a reverse transcriptase to generate complementary DNA (cDNA),
(ii) contacting said cDNA with sense and antisense primers that are specific for one of tumor necrosis factor super family 15, chemokine CXCL3, and interleukin 6 and a DNA polymerase to generate amplified DNA;

(d) comparing an expression level in portion (i) with the expression level in portion (ii); and

(e) predicting presence of the cytotoxic activity when the expression level in portion (i) is higher than the expression level in portion (ii). In several embodiments, the generation of cDNA comprises addition of primers (e.g., oligo dT primers) during the isolation of the RNA from the biological sample (e.g., primers are included in a lysis buffer used to liberate RNA from the biological sample). In several embodiments, the generation of cDNA comprises addition of primers after the isolation of the RNA from the biological sample and concurrently with the reverse transcriptase. In several embodiments, the generation of cDNA comprises addition of primers followed by an incubation period to allow annealing of the primers to the RNA from the biological samples, followed by the addition of a reverse transcriptase.

### Effects of the Invention

The present invention provides a method and means for predicting a cytotoxic activity (in particular, an ADCC activity) by detecting expression of a specific gene. Since the method and means in accordance with the present invention permit prediction of the cytotoxic activity only by a simple operation, that is, by detecting gene expression, they permit prediction of the cytotoxic activity in a simple and rapid manner with high accuracy without posing conventional problems, and therefore they are useful in many fields (particularly in clinical field).

By predicting the cytotoxic activity (in particular, an ADCC activity) according to the present invention, it is possible to evaluate the drug responsiveness of a patient to an antibody drug, which is useful particularly in a clinical field. In addition, the prediction of the cytotoxic activity according to the present invention permits screening for an antibody having the cytotoxic activity, and is useful in the fields of searching and development of antibody drugs.

### DESCRIPTION OF THE PREFERRED EMBODIMENT

The present inventors have succeeded in specifying a gene which shows a change in an expression level in response to antibody stimulation in correlation with the cytotoxic activity. Therefore, in the present invention, by measuring the change in the expression level of the gene in response to antibody stimulation, the cytotoxic activity, in particular, the antibody-dependent cellular cytotoxic activity (ADCC activity) can be predicted.

In the present invention, the “cytotoxic activity” is also referred to as cytotoxicity or cytotoxic activity. The “cytotoxic activity” signifies a property or an action providing effects such as death, functional disorder, or proliferation disorder on cells by destruction of substance or structure constituting a cell, inhibition to activities essential to living of a cell, effects on the cell cycle or the intracellular signal transmission, or the like. One of the “cytotoxic activities” includes an antibody-dependent cellular cytotoxic activity (ADCC activity). The “antibody-dependent cellular cytotoxic activity” or “ADCC activity” signifies a cytotoxic activity induced in an antibody-depending manner when an antibody bound to a target cell is bound to an Fc receptor on an effector cell (NK cell, macrophage, etc.). This technical term is sufficiently understood in this technical field.

The prediction of the cytotoxic activity in accordance with the present invention is carried out by preparing a biological sample including a leukocyte, bringing the biological sample and an antibody into contact with each other, detecting expression of at least one selected from the group consisting of tumor necrosis factor super family 15 (TNFSF15), chemokine CXCL3 (CXCL3), and interleukin 6 (IL-6) in the leukocyte, and comparing an expression level in a case where the sample is brought into contact with the antibody and a case where the sample is not brought into contact with the antibody. Herein, the “expression level” is an indicator showing a degree of expression of the subject gene. The “expression level” is represented by, for example, an expression level of the subject gene itself and a produced amount of protein encoded by the subject gene. In some embodiments, expression is determined by measuring an expression level of the gene itself, or a produced amount of protein (or activity of a protein) encoded by the subject gene. Thus, in some embodiments, the expression level is measured by, for example, reverse-transcription polymerase chain reaction (RT-PCR), real-time RT-PCR, or northern blotting, In one embodiment, the quantifying comprises amplifying RNA encoding the subject gene using RT-PCR. In some embodiments, expression level is measured by Western blotting, quantitative immunocytochemistry, ELISA, antibody array, fluorescence activated cell sorting, mass spectrometry, and/or protein activity assays.

In the method in accordance with the present invention, firstly, a biological sample including a leukocyte is prepared. The biological sample is not particularly limited as long as the leukocyte is included, and examples of the sample include blood (for example, whole blood), body fluid (for example, urine, cerebrospinal fluid, and ascites fluid), tissue, or the like. It is preferable that blood is used as the biological sample from the viewpoint of easy collection and the amount of the leukocyte present. Such biological samples can be prepared according to methods known in this technical field. A collection source is not particularly limited as long as it is any animal whose cytotoxic activity is to be predicted, and examples thereof include mammalian, for example, human, mouse, rat, monkey, cow, and horse, and human is preferred. The collected biological sample may be used as it is, or a fraction thereof including the leukocyte may further be isolated to be used.

Antibodies to be brought into contact are not particularly limited as long as they can be bound to an Fc receptor on an effector cell. Since the antigen-antibody reaction occurs specific to animal species in many cases, the antibody to be used is required to be reactive with respect to an animal as the collection source of the leukocyte. A person skilled in the art can easily understand the selection of such antibodies. For example, when it is intended to be brought into contact with a biological sample collected from a human patient, it is preferable to use human-derived antibodies, or chimeric antibodies or humanized antibodies. Examples of antigen-nonspecific antibodies that can be used include immunoglobulin IgA, IgM, IgD, IgE and IgG (IgG1, IgG2, IgG3, and IgG4, and the like). When the cytotoxic activity is predicted by using the antigen-nonspecific antibodies, it is possible to predict a general cytotoxic activity of the animal individuals or patients from which the biological sample (that is, leukocyte) is derived. Antibodies specific to a certain antigen, for example, known antibody drugs (anticancer drugs, antiviral agents, antiinflammatory agents, rejection inhibitors, antitumor drugs, and the like) may be used. Antibody drugs which are currently approved by US Food and Drug Administration (FDA) are listed in the following Table 1.

TABLE 1

| Antibody | | Types of | |
|---|---|---|---|
| name | Drug name | antibody | Target/indication |
| Abciximab | ReoPro | Chimeric | Glycoprotein (GP) IIb/IIIa |
| | (registered | antibody | Acute cardiac ischemic |
| | trademark) | | complication, unstable |
| | | | angina pectoris, thrombosis |
| Adalimumab | Humira | Human | TNF-α |
| | (registered | antibody | Inflammation (rheumatoid |
| | trademark) | | arthritis), psoriasis |
| Alemtuzumab | Campath | Humanized | CD52 |
| | (registered | antibody | Chronic lymphocytic |
| | trademark) | | leukemia (CLL) |
| Basiliximab | Simulect | Chimeric | IL-2 receptor α |
| | (registered | antibody | Inhibition of rejection |
| | trademark) | | |
| Bevacizumab | Avastin | Humanized | Vascular endothelial cell |
| | (registered | antibody | growth factor (VEGF) |
| | trademark) | | Large intestinal cancer, |
| | | | non-small cell lung cancer, |
| | | | breast cancer |
| Cetuximab | Erbitux | Chimeric | Epidermal growth factor |
| Daclizumab | (registered | antibody | receptor (EGFR) |
| | trademark) | | Large intestinal cancer, |
| | | | head and neck cancer |
| Daclizumab | Zenapax | Humanized | CD52 |
| | (registered | antibody | Inhibition of rejection |
| | trademark) | | |
| Eculizumab | Soliris | Humanized | Complement system protein |
| | (registered | antibody | C5 Paroxysmal nocturnal |
| | trademark) | | hemoglobinuria |
| Efalizumab | Raptiva | Humanized | CD11 |
| | (registered | antibody | A psoriasis |
| | trademark) | | |
| Gemtuzumab | Mylotarg | Humanized | CD33 |
| ozogamicin | (registered | antibody | Acute myelomonocytic |
| | trademark) | | leukemia (AML) |
| Ibritumomab | Zevalin | Mouse | CD20 |
| tiuxetan | (registered | antibody | Non-Hodgkin lymphoma |
| | trademark) | | (NHL) |
| Infliximab | Remicade | Chimeric | TNF |
| | (registered | antibody | Inflammation (rheumatoid |
| | trademark) | | arthritis) |
| Muromonab | Orthoclone | Mouse | CD3 receptor |
| | (registered | antibody | Inhibition of rejection |
| | trademark) | | |
| Natalizumab | Tysabri | Humanized | VLA-4 receptor |
| | (registered | antibody | Multiple sclerosis, Crohn's |
| | trademark) | | disease |
| Omalizumab | Xolair | Humanized | IgE |
| | (registered | antibody | Asthma |
| | trademark) | | |
| Palivizumab | Synagis | Humanized | RSV F protein epitope |
| | (registered | antibody | Virus infection |
| | trademark) | | |
| Panitumumab | Vectibix | Human | Epidermal growth factor |
| | (registered | antibody | receptor (EGFR) |
| | trademark) | | Large intestinal cancer |
| Ranibizumab | Lucentis | Humanized | Vascular endothelial cell |
| | (registered | antibody | growth factor (VEGF) |
| | trademark) | | Large intestinal cancer, |
| | | | non-small cell lung cancer, |
| | | | aged macular degeneration |
| Rituximab | Rituxan | Chimeric | CD20 |
| | (registered | antibody | Non-Hodgkin lymphoma |
| | trademark), | | (NHL) |
| | Mabthera | | |
| | (registered | | |
| | trademark) | | |
| Tositumomab | Bexxar | Mouse | CD20 |
| | (registered | antibody | Non-Hodgkin lymphoma |
| | trademark) | | (NHL) |
| Tocilizumab | Tocilizumab | Humanized | IL-6 receptor |
| | (registered | antibody | Autoimmune disease |
| | trademark) | | |
| Golimumab | Simponi | Human | TNF-α |
| | (registered | antibody | Autoimmune disease |
| | trademark) | | |
| Canakinumab | llaris | Human | IL-1b |
| | (registered | antibody | Inflammation |
| | trademark) | | |
| Ustekinumab | Stelara | Human | IL-12/IL-23 |
| | (registered | antibody | Autoimmune disease |
| | trademark) | | |
| Ofatumumab | Arzerra | Human | CD20 |
| | (registered | antibody | Cancer |
| | trademark) | | |
| Denosumab | Prolia | Human | RANK ligand |
| | (registered | antibody | Osteoporosis |
| | trademark) | | |
| Motavizumab | Numax | Humanized | RSV |
| | (registered | antibody | Anti-infection |
| | trademark) | | |
| Raxibacumab | ABThrax | Human | Anthrax toxin |
| | (registered | antibody | Anti-infection |
| | trademark) | | |
| Belimumab | Benlysta | Human | BLyS (B-cell activation |
| | (registered | antibody | factor) |
| | trademark) | | Autoimmune disease |
| Ipilimumab | Yervoy | Human | CTLA-4 |
| | (registered | antibody | Cancer |
| | trademark) | | |
| Brentuximab | Adcetris | Chimeric | CD30 |
| vedotin | (registered | antibody | Cancer |
| | trademark) | | |
| Trastuzumab | Herceptin | Humanized | Her2/neu |
| | (registered | antibody | Breast cancer |
| | trademark) | | |

Antibodies, such as, for example, those listed in Table 1, can be administered by protocols established by the FDA. For example, administration may be, depending on the embodiment, can be intradermal, subcutaneous, via a transdermal implant, intravenous, intramuscular, intraperitoneal, intraarterial, intracavernous, intracerebral, intrathecal, epidural, and the like.

An antibody, in which an Fc region thereof is bound to the Fc receptor, activates an effector cell and exhibits a cytotoxic activity (that is, an ADCC activity). Therefore, it is preferable that the antibody is brought into contact with a leukocyte in a state in which the Fc region is exposed.

In the present invention, it is preferable to use heat-aggregated IgG (HAG) as an antibody. This is because HAG is a model of an immune complex including all subclasses such as IgG1, IgG2, IgG3, and IgG4, and the cytotoxic activity (that is, an ADCC activity) against HAG can be said to be a cytotoxic activity on any types of antibodies. HAG can be prepared according to methods known in the technical field. For example, HAG may be prepared by heating human IgG at 63° C. for 15 min (Ostreiko, K. K. et al., Immunol. Lett. 15: 311-316, 1987). The prepared HAG may be stored at −20° C. before use.

Next, a biological sample including a leukocyte and an antibody are brought into contact with each other. In the present invention, “contact” means that an antibody and a leukocyte are brought into close contact with each other such that the antibody activates the leukocyte included in the biological sample. For example, it includes operations of mixing a biological sample with a solution containing an antibody (an antibody-containing solution), adding an antibody-containing solution to a biological sample, immersing a biological sample into an antibody-containing solution, or the like. The contact is carried out at, for example, 4 to 42° C. (preferably, at about 37° C.) for 1 min to 24 hours (preferably, for about 2 to 8 hours).

Subsequently, expression of at least one gene or protein (collectively, a “marker”) selected from the group consisting of tumor necrosis factor super family 15 (TNFSF15), chemokine CXCL3 (CXCL3), and interleukin 6 (IL-6) in the leukocyte is detected. In the present specification, these genes are also referred to as generically “subject gene.” The “detection” herein denotes not only measurement of the expression level of the subject gene as an absolute amount but also measurement as a relative amount or ratio.

TNFSF15 is a member of the TNF family, and is known to be bound to its receptor TNFRSF25 to induce apoptosis (Kitson, J. et al. Nature 384: 372-375, 1996). The gene sequence of TNFSF15 is registered in GenBank in which the nucleotide sequence of mRNA is registered under the accession number of NM_{—}005118, and the nucleotide sequence of genome DNA including the nucleotide sequence of TNFSF15 is registered under the accession number of NG_{—}011488. CXCL3 is one type of chemokines, but the function thereof is not sufficiently elucidated. The gene sequence of CXCL3 is registered in GenBank in which the nucleotide sequence of mRNA is registered under the accession number of NM_{—}002090, and the nucleotide sequence of genome DNA including the nucleotide sequence of CXCL3 is registered under the accession number of NC_{—}000004. IL-6 is one type of inflammatory cytokines. The gene sequence of IL-6 is registered in GenBank in which the nucleotide sequence of mRNA is registered under the accession number of NM_{—}000600, and the nucleotide sequence of genome DNA including the nucleotide sequence of IL6 is registered under the accession number of NG_{—}011640. CXCL1 is said to function as a chemoattractant in the neutrophil, and to be involved in the inflammation reaction or the like. The gene sequence of CXCL1 is registered in GenBank in which the nucleotide sequence of mRNA is registered under the accession number of NM_{—}001511, and the nucleotide sequence of genome DNA including the nucleotide sequence of CXCL1 is registered under the accession number of NC_{—}000004. TNFSF2 is a member of the TNF family, and is bound to its receptors TNFR1A and TNFR1B so as to induce apoptosis or cause inflammation. The gene sequence of TNFSF2 is registered in GenBank in which the nucleotide sequence of mRNA is registered under the accession number of NM_{—}000594, and the nucleotide sequence of genome DNA including the nucleotide sequence of TNFSF2 is registered under the accession number of NG_{—}007462.

The expression of the subject gene may be detected at a gene level or protein level, any of which may be carried out by using techniques known to a person skilled in the art. From the viewpoint that it takes a long time to produce protein responding to antibody stimulation for the detection at the protein level, detection of the expression at the gene level is preferable. Note here that it is preferable that a leukocyte is isolated and dissolved before detecting the expression, but when the used biological sample is a fraction including only a leukocyte, the biological sample can be used as it is. Isolation and dissolution of the leukocyte may be carried out by using methods known in the technical field.

Examples of the method for detecting the expression at the gene level include a method for detecting mRNA or corresponding cDNA of the subject gene by using a primer or a probe. Specifically, by purifying mRNA from the total RNA of the leukocyte, and amplifying mRNA specific to the subject gene by using a primer, the amplified product may be detected (RT-PCR method). Alternatively, from total RNA or mRNA, or cDNA synthesized based on the total RNA or mRNA, total RNA or mRNA, or cDNA specific to the subject gene may be detected by using a probe (hybridization method).

The total RNA or the mRNA may be prepared by using methods known in the technical field. For example, the total RNA may be prepared by guanidine-cesium chloride ultra-centrifugal method, AGPC (acid quanidium-phenol-chloroform) method, or the like, and the mRNA may be isolated by using oligo dT. The synthesis of the cDNA from the total RNA or the mRNA may be carried out by using reverse transcriptase, which is also known in the technical field. These methods may be carried out by using a commercially available kit in a simple and easy manner.

Primers or probes may be designed based on the nucleotide sequence of the subject gene according to means known to a person skilled in the art. For designing the primers or probes, the following points are known to be considered. The length which functions as the primer is preferably 10 bases or more, more preferably 15 to 50 bases, and further more preferably 20 to 30 bases. The length which functions as the probe is preferably 10 bases or more, more preferably 15 to 50 bases, and further more preferably 20 to 30 bases. Whether or not the melting temperatures (Tm) of the primer or the probe are appropriate is determined. For determining Tm, the known software for designing primers or probes may be used, examples of the software usable in the present invention include Primer Express (registered trademark) (Applied Biosystems, FOster City, Calif.), HYBsimulator (trademark) (RNAture, Irvine, Calif.), and the like. The conditions under which annealing or hybridization specific to the subject gene as the primer or the probe is possible include GC content, which is well known to a person skilled in the art.

In the present invention, for example, the following primer sequence may be used.

TABLE 2

| Name of | | |
|---|---|---|
| gene | Forward primer | Reverse primer |
| TNFSF15 | TGCGAAGTAGGTAGCAACT | CCATTAGCTTGTCCCCTTCTT |
| | GGTT | G |
| | (SEQ ID No. 1) | (SEQ ID No. 2) |
| CXCL3 | GGAATTCACCTCAAGAACAT | GTGGCTATGACTTCGGTTTG |
| | CCA | G |
| | (SEQ ID No. 3) | (SEQ ID No. 4) |
| IL-6 | TCATCACTGGTCTTTTGGAG | TCTGCACAGCTCTGGCTTGT |
| | TTTG | (SEQ ID No. 6) |
| | (SEQ ID No. 5) | |
| CXCL1 | CCACTGCGCCCAAACC | GCAGGATTGAGGCAAGCTTT |
| | (SEQ ID No. 7) | (SEQ ID No. 8) |
| TNFSF2 | GGAGAAGGGTGACCGACTC | TGCCCAGACTCGGCAAAG |
| | A | (SEQ ID No. 10) |
| | (SEQ ID No. 9) | |

The designed primers and probes as mentioned above can be prepared according to methods known to a person skilled in the art. Furthermore, as is well known to a person skilled in the art, the primer or the probe may include added sequences such as tag sequence which is a sequence except for a part to be annealed or hybridized. The primer or the probe may be fixed to appropriate solid phase such as a filter, a membrane, a slide glass, and a microtiter plate.

In order to detect the expression of the subject gene in the leukocyte, by using the above-mentioned primer or the probe for an amplification reaction or hybridization, the amplified product or the hybridized product is detected.

When amplification is carried out from mRNA specific to the subject gene by using a primer, arbitrary amplification means can be used. Examples of the method include known methods using a principle of the polymerase chain reaction (PCR) method. When quantitative PCR methods such as a competitive PCR method and a real time PCR method may be employed as the amplification method, quantitative detection can be carried out. A person skilled in the art can easily decide the optimum conditions of the amplification reaction.

For detection of the amplified product, for example, a method for introducing a labeling substance such as radioisotope, fluorescent substance, and luminescent substance into dNTP incorporated in the process of amplification reaction and detecting the labeling substance. Examples of the radioisotope to be used include ³²P, ¹²⁵I, ³⁵S, and the like. Examples of the fluorescent substance to be used include SYBR Green and fluorescein (FITC). Examples of the luminescent substance to be used include luciferin, or the like. The labeling substance may be introduced by using a method which is known in the technical field or commercially available kits. Detection of labeling substance incorporated into the amplified product can be carried out by using a method known in the technical field. For example, when the radioisotope is used as the labeling substance, the radioactivity may be measured by using, for example, liquid scintillation counter, γ-counter, or the like. When the fluorescent substance is used as the labeling substance, fluorescence of the fluorescent substance may be detected by using a fluorescence microscope, fluorescence plate reader, or the like.

By carrying out hybridization of the total RNA or mRNA, or cDNA synthesized from them by using a probe, and by detecting the specific binding (hybrid) thereof, the expression of the subject gene can be detected. The hybridization is required to be carried out under conditions such that the probe is specifically bound to only RNA or DNA derived from the subject gene, that is, under stringent conditions. Such stringent conditions are well known in the technical field. Examples of the stringent conditions include conditions described in J. Sambrook et al., Molecular Cloning, A Laboratory Manual, Third Edition, Cold Spring Harbor Laboratory Press (2001), item 8 of Chapter 7 “Northern Hybridization,” or the like. More specific examples of the conditions include a condition that hybridization is carried out at about 45° C. and at 6.6×SSC, followed by washing at 50° C. and at 2.0×SSC. For selecting the stringency, the salt concentration in the washing step can be selected from, for example, low stringency at about 2.0×SSC at 50° C. to high stringency at about 0.2×SSC at 50° C. Furthermore, the temperature in the washing step can be increased from room temperature in the low stringency condition, that is, about 22° C., to about 65° C. in the high stringency condition.

When the hybridization is carried out, it is possible to detect a hybridization product by adding, to a probe, appropriate labeling substances such as fluorescent labels (fluorescein, rhodamine, or the like), radioactive labels (³²P or the like), enzyme labels (alkaline phosphatase, horseradish peroxidase, or the like), and biotin labels.

As mentioned above, expression of the subject gene can be detected by detecting products by the amplification reaction or hybridization by using a primer or a probe.

The study group of the present inventors has developed a system (Hem(A)⁺ system) capable of quantifying slight change in the expression of the gene (gene expression) in the leukocyte in a simple and rapid manner (U.S. Pat. No. 6,844,158, U.S. Pat. No. 7,258,976, WO99/32654, U.S. Pat. No. 7,745,180 corresponding to U.S. patent application Ser. No. 10/698,967 and U.S. patent application Ser. No. 10/796,298, WO03/091407, Mitsuhashi, M. et al., Clin. Chem. 52: 634-642, 2006, etc). In the present invention, such a system can be used.

The Hem(A)⁺ system is described simply. Firstly, the whole blood is added to a filter plate so as to capture the leukocyte. Herein, a step of bringing it into contact with an antibody in the method according to the present invention may be carried out by using the whole blood as it is, or may be carried out after the leukocyte is captured. From the viewpoint of reactions under conditions similar to the living body, it is preferable that the whole blood and the antibody are brought into contact with each other, followed by capturing the leukocyte. Next, the leukocyte is dissolved on the filter plate, and the standard RNA and an antisense (reverse) primer with respect to the subject gene are added. The obtained cell dissolution solution is transferred to an oligo dT solid phase plate, and subjected to hybridization. The mRNA captured on the oligo dT solid phase plate is reverse transcribed by an antisense primer included in a cell dissolution solution, and thus the corresponding cDNA is synthesized. Based on the amount of the synthesized cDNA, the expression level of the subject gene in the leukocyte can be obtained. See the detail thereof in the above-mentioned documents.

Examples of methods for measuring a protein level include a method for detecting protein encoded by the subject gene by using, for example, an antibody. The detection method using an antibody is known in the technical field, and examples of the method include an enzyme immunoassay (EIA), an enzyme linked immunosorbent assay (ELISA), a fluorescence immunoassay, a radioactive immunoassay (RIA), an immunoprecipitation method, a western blotting method, or the like. The detection of protein using an antibody may be carried out according to, for example, the description in Short Protocols in Molecular Biology, Chapter 11 “immunology” John Wiley & Sons, Inc. 1995 edited by Ausubel, F. M. et al.

The antibody to be used for detection is a polyclonal antibody or a monoclonal antibody. The above-mentioned antibody is a whole molecule or fragment or the like that can be bound to protein epitope encoded by each subject gene. Such an antibody, for example, when it is a polyclonal antibody, may be obtained from the serum after antigen polypeptide or a partial fragment thereof is immunized into an animal as an immunogen. Alternatively, an expression vector into which the subject gene or a partial sequence thereof is inserted by injection or a gene gun can be produced by being introduced into animal muscle or skin, and then collecting the serum. Examples of the animals to be immunized include a mouse, a rat, a rabbit, a goat, a chicken, or the like. The monoclonal antibody may be produced according to the known monoclonal antibody production method (“Monoclonal Antibody,” Komei Nagamune and Hiroshi Terada, Hirokawa Shoten, 1990; “Monoclonal Antibody” James W. Goding, third edition, Academic Press, 1996).

The binding of the target protein and the antibody may be measured according to the well-known methods. A person skilled in the art can determine an effective and optimum measurement method according to the types and forms of the immunoassay to be employed, types of labeling substances to be used, subjects of labels, and the like. For example, for easily detecting the reaction between protein encoded by the subject gene in the leukocyte and an antibody against it, the reaction can be directly detected by labeling the antibody with a labeling substance, or can be indirectly detected by using a labeling secondary antibody or biotin-avidin complex or the like. Such labels are also known in the technical field, and in the case of the enzyme immunoassay, for example, peroxidase, β-galactosidase, alkaline phosphatase, may be used. In the case of the fluorescence immunoassay, for example, fluorescein isothiocyanate (FITC) may be used. In the case of the radioactive immunoassay, for example, tritium and iodine¹²⁵ may be used. Detection of such labeling substances can be carried out according to the methods known in the technical field. The antibody may be fixed to a solid phase (a membrane, a filter, a bead, a plate, or the like) (solid phase system), and may be used as a solution (liquid phase system).

When the antibody is made to be a labeled antibody by directing labeling with, for example, a labeling substance, a sample prepared from the leukocyte is brought into contact with the labeled antibody such that the target protein and the antibody are bound to each other. Then, by separating an unbound labeled antibody, it is possible to measure the expression level of the subject gene in the leukocyte from the amount of the bound labeled antibody or the amount of the unbound labeled antibody. For example, in the case where the labeled secondary antibody is used, the antibody and a sample prepared from the leukocyte are reacted to each other (primary reaction), and then the obtained complex is further reacted with the labeled secondary antibody (secondary reaction). By separating the unbound labeled secondary antibody, it is possible to measure the expression level of the subject gene in the leukocyte from the amount of the bound labeled secondary antibody or the amount of the unbound labeled secondary antibody.

As mentioned above, after the expression of the subject gene is detected, the expression level in the case where the sample is brought into contact with the antibody and the expression level in the case where the sample is not brought into contact with the antibody are compared with each other. When the expression in the case where the sample is brought into contact with the antibody is higher than the expression level in the case where the sample is not brought into contact with the antibody, a cytotoxic activity is present. Specifically, from the ratio of the expression level with respect to the case where the sample is brought into contact with the antibody to the case where the sample is not brought into contact with the antibody, the cytotoxic activity can be predicted. For example, when the ratio of the gene expression level in the case where the sample is brought into contact with the antibody is 1.2 or more, and preferably 2.0 or more wherein the gene expression level in the case where the sample is not brought into contact with the antibody is defined as 1, it is predicted that the cytotoxic activity is present.

The present invention also relates to a kit for predicting the cytotoxic activity, in particular, the ADCC activity. Such a kit includes means for detecting expression of at least one selected from the group consisting of tumor necrosis factor super family 15 (TNFSF15), chemokine CXCL3 (CXCL3), and interleukin 6 (IL-6). Such means for detecting the expression is not particularly limited as long as it is means capable of detecting the expression of the subject gene at a gene level or a protein level as mentioned above, and examples thereof include a primer or a probe, or an antibody. The kit may include other components useful for detecting the expression of the subject gene. For example, the kit may include a reagent for preparing a biological sample, a buffer, a labeling substance, a reaction solution, an instruction, and the like. The use of such kits makes it easier to predict the cytotoxic activity in accordance with the present invention.

Since the cytotoxic activity (the ADCC activity) represents the cytotoxicity of the antibody on biological cells, the present invention may be applied for various applications of use in which an indicator is the cytotoxic activity.

For example, the cytotoxic activity shows drug responsiveness of a patient to an antibody drug (for example, Gennari, R. et al., Clin. Cancer Res. 10: 5650-5655, 2004; Beano, A. et al., Journal of Translational Medicine 6: 25, 2008). Therefore, drug responsiveness of a patient to an antibody drug can be evaluated by predicting the cytotoxic activity in a patient. Specifically, firstly, a biological sample including a leukocyte from a patient is prepared, and then the biological sample and an antibody are brought into contact with each other. Next, the expression of at least one selected from TNFSF15, CXCL3, CXCL1 and TNFSF2 in the leukocyte is detected, and the expression level in the case where the sample is brought into contact with the antibody and the expression level in the case where the sample is not brought into contact with the antibody are compared with each other so as to predict the cytotoxic activity. At this time, when the cytotoxic activity of the patient is high, it is evaluated that the drug responsiveness of the patient to the antibody drug is high. In other words, when the expression level in the case where the sample is brought into contact with the antibody is higher than the expression level in the case where the sample is not brought into contact with the antibody, it is evaluated that the drug responsiveness of the patient to the antibody drug is higher. For example, when the ratio of the gene expression level in the case where the sample is brought into contact with the antibody is 1.2 or more, and preferably 2.0 or more wherein the gene expression level in the case where the sample is not brought into contact with the antibody is defined as 1, it is predicted that the cytotoxic activity of the patient is high and that the drug responsiveness of the patient to the antibody drug is high. The antibody with which the biological sample is brought into contact may be an antibody drug to be administered to a patient, or an antigen-nonspecific antibody such as HAG.

As one example, evaluation of the drug responsiveness of a patient to trastuzumab (trade name: Herceptin (registered trademark)) is described. The patient is a breast cancer patient who may have or may not have already undergone treatment with trastuzumab and/or other treatment (surgical operation, radiation therapy, chemotherapy, immunotherapy, or the like). The patients may include patients with a primary breast cancer and/or a metastatic breast cancer, and furthermore, a cancer that has metastasized from breast cancer to other tissue. A biological sample including a leukocyte (for example, whole blood) from the patient is prepared, and the sample is brought into contact with an antibody. Antibodies to be brought into contact may be an antigen-nonspecific antibody (for example, HAG) or trastuzumab itself. After the sample is brought into contact with an antibody, expression of at least one of TNFSF15, CXCL3, CXCL1 and TNFSF2 in the leukocyte is detected. Thereafter, the expression level in the case where the sample is brought into contact with the antibody and the expression level in the case where the sample is not brought into contact with an antibody are compared with each other. When the expression in the case where the sample is brought into contact with the antibody is significantly higher than the expression level in the case where the sample is not brought into contact with an antibody, it can be evaluated that the drug responsiveness of the patient to trastuzumab (or to antibody drugs as a whose when an antigen-nonspecific antibody is used) is high. For example, when the ratio of the gene expression level in the case where the sample is brought into contact with the antibody is 1.2 or more, and preferably 2.0 or more wherein the gene expression level in the case where the sample is not brought into contact with the antibody is defined as 1, it can be evaluated that the drug responsiveness of the patient to trastuzumab (or to antibody drugs as a whose when an antigen-nonspecific antibody is used) is high.

At present, the drug responsiveness to trastuzumab is evaluated based on the presence or absence of expression of a human epidermal growth factor receptor type 2 (HER2 protein) antigen and subjects to be administered are determined. However, there are many patients who do not have responsiveness to trastuzumab. Therefore, in the present invention, the drug responsiveness of a patient to trastuzumab can be evaluated in combination with the prediction of the cytotoxic activity and the presence or absence of the expression of the HER2 antigen (or a Her2/neu gene thereof). The detection of the expression of the HER2 antigen can be carried out by an immunohistochemical staining (IHC) method, a fluorescent in situ hybridization (FISH) method, or the like, which are known in the technical field, and detection kits are also marketed (for example, Hercep Test based on the IHC method, manufactured by Dako). In general, it is evaluated that a patient who expresses excessive HER2 antigen has high drug responsiveness to trastuzumab.

It can be also said that the cytotoxic activity (the ADCC activity) shows an effect of an antibody drug itself. Therefore, when new antibody drugs are developed, or existing antibody drugs are modified, the cytotoxic activity of the antibody is predicted according to the present invention, and it is possible to carry out screening for an antibody having the cytotoxic activity, and preferably having a high cytotoxic activity. Specifically, firstly, a subject antibody is prepared, and the antibody is brought into contact with the leukocyte. The leukocyte is derived from an animal which is intended to be tested for the effect of the subject antibody. The leukocyte may be isolated or may be a sample including the leukocyte (for example, whole blood sample). After the leukocyte is brought into contact with the subject antibody, the expression of at least one of TNFSF15, CXCL3 and IL-6 in the leukocyte is detected. As a result, the expression level in the case where the leukocyte is brought into contact with the subject antibody is higher than the expression level in the case where the leukocyte is not brought into contact with the subject antibody, the subject antibody can be selected to be one having the cytotoxic activity. For example, when the ratio of the gene expression level in the case where the leukocyte is brought into contact with the antibody is 1.2 or more, and preferably 2.0 or more wherein the gene expression level in the case where the leukocyte is not brought into contact with the antibody is defined as 1, the subject antibody can be selected to be one having the cytotoxic activity.

### BRIEF DESCRIPTION OF THE DRAWINGS

FIG. 1 is a graph showing a correlation between an expression level of genes of TNFSF15, CXCL3 and IL-6 and an ADCC activity.

FIG. 2 is a graph showing a correlation between therapeutic effect by preoperative chemotherapy to a breast cancer patient and the expression level of genes of TNFSF15, CXCL3, CXCL1 and TNFSF2.

### DESCRIPTION OF PREFERABLE EXAMPLES

### Example 1

Hereinafter, the present invention is further described with reference to Examples, but the Examples are not intended to limit the present invention but exemplify the same.

Samples and Reagents

As a sample, blood from eight healthy individuals was used. Blood was collected by using a vacuum blood-collecting vessel (manufactured by TERUMO CORPORATION, trade name VENOJECT (registered trademark) II vacuum blood-collecting vessel containing heparin sodium). As an antibody to which the collected blood (hereinafter, also referred to as simply “blood”) is reacted, Human IgG Purified Immunoglobulin (manufactured by Sigma-Aldrich Co. LLC, product number I 4506) was used. The above-mentioned antibody was dissolved in PBS(−) (manufactured by Wako Pure Chemical Industries, Ltd., product No. 531-16615,) so that the concentration was 20 mg/mL. The above-mentioned dissolved antibody solution (antibody solution) was heated at 63° C. for 30 min, and then the above-mentioned PBS(−) was further added to be prepared so as to be 5 mg/mL. In the obtained solution, the heat-aggregated antibody was dissolved. The solution was made to be a heat-aggregated IgG solution.

Reaction between Sample and Reagent For reaction between the above-mentioned blood and the above-mentioned heat-aggregated IgG solution, a 200 μL-volume microtube was used. In the tube, 70 μL of blood and 2.8 μL of the above-mentioned PBS(−) were mixed by using suction discharge of a pipette mildly. In another tube, 70 μL of blood and 2.8 μL of 5 mg/mL heat-aggregated IgG solution were mixed by using suction discharge of a pipette mildly. They were carried out under each condition such that n=3 was obtained. A mixed solution of the blood and the heat-aggregated IgG solution and a mixed solution of the blood and the PBS(−) were stood still in thermostat at 37° C. for four hours. Hereinafter, the above-mentioned mixed solution may be a blood sample.

Purification of mRNA and Synthesis of cDNA

From the blood sample which had stood still for four hours and incubated, 50 μL of the sample was taken out and used for purification of mRNA (mRNA purification) and synthesis of cDNA (cDNA synthesis). For purification of mRNA and synthesis of cDNA, the Hem(A)⁺ system described in Mitsuhashi M. et. al., ClinChem 52,4: 634-642, 2006 was used so as to obtain a sample of cDNA (cDNA sample) from the above-mentioned blood sample.

Measurement of Expression level of Gene

The above-obtained cDNA was used as a sample, and the expression level of the gene (gene expression level) was measured by the quantitative PCR method. For the detection by the quantitative

PCR method, the detection method by SYBRgreen was used. As a detection reagent, iTaq SYBRgreen Supermix with ROX (manufactured by Bio-Rad Laboratories, Inc., product number 172-5853) was used. As the primer, primers having respective sequences shown in SEQ ID NOs 1 to 6 were prepared so that the respective final concentration was 500 nM and used. As equipment used for the quantitative PCR method, ABIPRISM7900HT real time PCR system (trade name, manufactured by Applied Biosystems) was used.

Measurement of ADCC Activity (Antibody-Dependent Cellular Cytotoxic Activity)

The ADCC activity was measured by a method using calcein. For the target cell, MCF7 cells were used. The above-mentioned MCF7 cells were cultured in 10 μg/mL Calcein AM solution at 37° C. for 30 min. For the effector cells, the peripheral blood mononuclear cell (PBMC) obtained from the blood was used. Hereinafter, four types of combinations of reaction systems were produced. The below-mentioned heat-treated PBMC was produced by heating PBMC at 56° C. for 30 min. The below-mentioned NP40 means polyoxyethylene (9) octylphenyl ether. (1) Experimental group (Experimental): PBMC (1×10⁵ cells)+MCF7 cells (1×10⁴ cells)+trastuzumab (final concentration: 5 μg/mL), (2) Spontaneous release group (Spontaneous release): heat-treated PBMC (1×10⁵ cells)+MCF7 cells (1×10⁴ cells)+trastuzumab (final concentration: 5 μg/mL), (3) Maximum release group (Maximum release): 0.1% by mass NP40+MCF7 cells (1×10⁴ cells), (4) Control group (Control): MCF7 cells (1×10⁴ cells)+trastuzumab (final concentration: 5 μg/mL)

Reaction by reaction systems of the above-mentioned (1) to (4) was carried out for four hours. The fluorescence intensity of supernatant obtained after respective reaction was measured (excitation: 490 nm/absorption 515 nm). Values indicating the ADCC activity (ADCC activity values) were calculated according to the formula: (fluorescence intensity of experimental group−fluorescence intensity of spontaneous release group)/(fluorescence intensity of maximum release group−fluorescence intensity of control group).

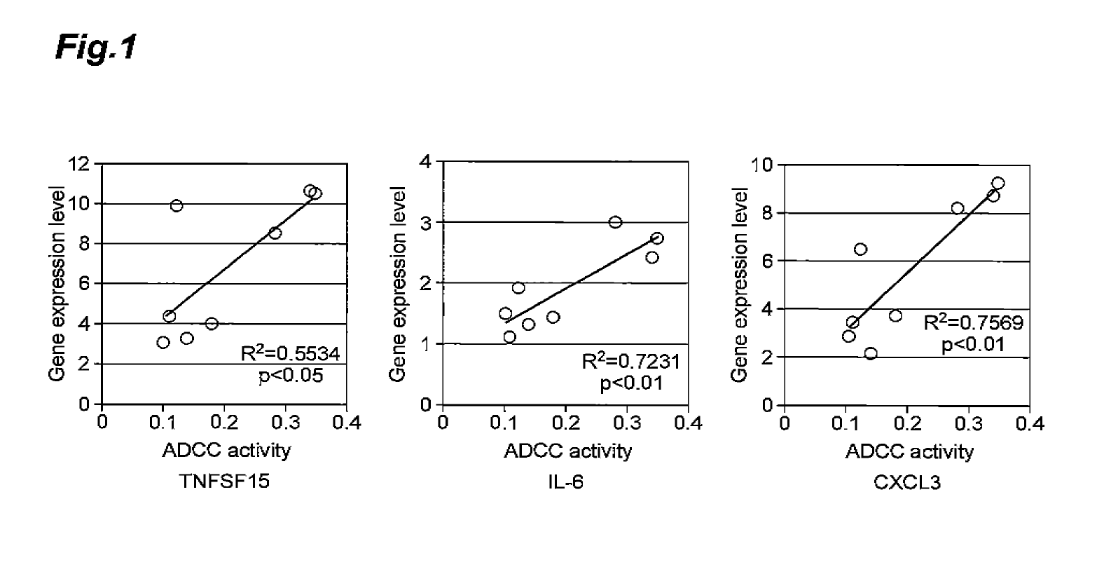

Gene Showing Behavior Correlating to ADCC Activity The correlation between the ADCC activity value calculated by the above-mentioned method and change in the gene expression level measured by the quantitative PCR method was observed. The correlation was shown in FIG. 1. In FIG. 1, X-axis shows the ADCC activity, and Y-axis shows a gene expression level measured by using the Hem(A)+ method. As a result, good correlation was obtained in three genes, TNFSF15, IL-6 and CXCL3.

### Example 2

Peripheral blood of a breast cancer patient was used as a sample, and the relation between a gene expression level of the leukocyte in the peripheral blood and the treatment result of preoperative chemotherapy was investigated. Firstly, a difference in the gene expression level between a case where a heat-aggregated IgG solution and the above-mentioned peripheral blood were brought into contact with each other and a case where only a solvent of the heat-aggregated IgG solution (PBS(−)) and the above-mentioned peripheral blood were brought into contact with each other was determined. Next, the relation between the obtained difference of the above-mentioned gene expression level and the treatment results of preoperative chemotherapy was compared with each other and investigated. In carrying out the comparison investigation as mentioned above, after the approval of carrying out investigation was given by Ethics Committee of the experiment facility, peripheral blood as blood samples were provided from patients who gave the consent to the investigation.

Cases to be Subjected

Cases to be subjected (subject cases) were breast cancer patients who satisfy all of the following five items of (1) to (5). (1) Cases in operable stages II to IIIA, and having a tumor diameter of more than 3 cm. (2) Cases determined to be positive by an immunohistochemical method (IHC method) in HER2 examination (HER2 staining intensity: 3+), or cases in which the ratio of the total number of HER2 signals to the total number of CEP17 signals in determination by the fluorescent in situ hybridization method (FISH method) is more than 2.0. (3) Cases which have undergone treatment using paclitaxel and trastuzumab, following the treatment with epirubicin, cyclophosphamide, and 5FU as the preoperative chemotherapy. (4) Cases in which a patient is 20 years old or more. (5) Cases having Performance Status of Eastern Cooperative Oncology Group of 0 to 2 (reference document: Oken, M. M., Creech, R. H., Tormey, D. C., Horton, J., Davis, T. E., McFadden, E. T., Carbone, P. P.: Toxicity And Response Criteria Of The Eastern Cooperative Oncology Group. Am J Clin Oncol 5:649-655, 1982).

The subject cases exclude cases with complication with other tumor, congestive heart disease, uncontrollable angina pectoris, arrhythmia, symptomatic infectious disease, serious diarrhea, symptom of hydropericardium, and symptom of brain metastasis.

Samples and Reagents

As the sample, blood from 18 breast cancer patients was used. Blood was collected by using a vacuum blood-collecting vessel (manufactured by TERUMO CORPORATION, trade name VENOJECT (registered trademark) II vacuum blood-collecting vessel containing heparin sodium) before carrying out preoperative chemotherapy. The antibody solution and the heat-aggregated IgG solution were the same as those used in Example 1.

Reaction Between Sample and Reagent

The reaction between a sample and a reagent was carried out by the same operation as in Example 1.

Purification of mRNA and Synthesis of cDNA

For purification of mRNA and synthesis of cDNA, the same operation as in Example 1 was carried out.

Measurement of Gene Expression level

The gene expression levels of TNFSF15, CXCL3, CXCL1 and TNFSF2 in each blood sample were measured by the same method as in Example 1 except that primers having the sequences shown in SEQ ID NOs 1 to 4 and 7 to 10 were used as the primer. By comparing the gene expression level in the case where the blood sample was brought into contact with the antibody (heat-aggregated IgG) and gene expression level in the case where the blood sample was not brought into contact with the antibody, the difference of the gene expression levels were obtained.

Determination of Therapeutic Effect of Patient

The therapeutic effect was determined by measuring increase and decrease of tumor lesion before and after the treatment by preoperative chemotherapy based on the effect that the tumor contracts (tumor contraction effect). Determination of the tumor contraction effect was carried out based on the RECIST guidelines (Response Evaluation Criteria in Solid Tumors, reference literature: Journal of the National Cancer Institute, 2000, Vol. 92, No. 3, 205-216). The criteria for determining RECIST include four ratings: complete response (CR), partial response (PR), progressive disease (PD), and stable disease (SD). In this Example, among determination according to the RECIST guidelines, cases showing complete response (CR), partial response (PR) were considered cases (pCR) in which therapeutic effect by preoperative chemotherapy was observed. As a result, the therapeutic effect was observed in 11 cases (pCR), and therapeutic effect was not observed in 7 cases (non-pCR).

Gene Related to Therapeutic Effect

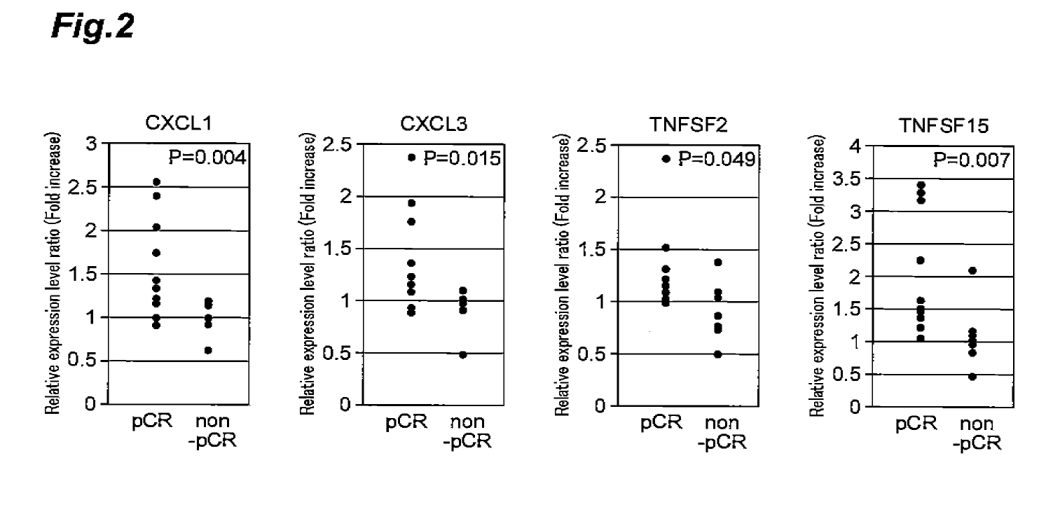

The relation between therapeutic effect determined as mentioned above and change in the gene expression level (difference in the gene expression level) measured by the quantitative PCR method was examined. The results thereof are shown in FIG. 2. In FIG. 2, X axis shows treatment result (pCR, non-pCR), and Y axis shows relative expression level ratio based on the measured gene expression level (Fold Increase) by using the Hem(A)+ method. The relative expression level ratio mentioned above is shown as the gene expression level in the case where the blood sample was brought into contact with the antibody wherein the gene expression level in the case where the blood sample was not brought into contact with the antibody was defined as 1. As a result, in four genes, that is, TNFSF15, TNFSF2, CXCL1, and CXCL3, significant difference was observed between the relative expression level ratio in the case where the therapeutic effect was observed (pCR) and the relative expression level ratio in the case where the therapeutic effect was not observed (non-pCR).

All the patents, patent applications, and documents cited herein are incorporated by reference in their entirety.

### INDUSTRIAL APPLICABILITY

The present invention provides a method and means for predicting a cytotoxic activity, in particular, an ADCC activity by detecting the expression of a specific gene. The method and means in accordance with the present invention permit prediction of the cytotoxic activity only by a simple operation, that is, by detecting gene expression. The method and means in accordance with the present invention permit prediction of the cytotoxic activity in a simple and rapid manner with high accuracy without posing conventional problems, and therefore they are useful in many fields (particularly in, clinical field).

By predicting the cytotoxic activity according to the present invention, it is possible to evaluate the drug responsiveness of a patient to the antibody drug, which is useful particularly in a clinical filed. In addition, the prediction of the cytotoxic activity according to the present invention permits screening for an antibody having the cytotoxic activity, and is useful in the fields of searching and development of an antibody drug.

What is claimed is:

1. A method for predicting antibody-dependent cellular cytotoxic (ADCC) activity in a subject, the method comprising the steps of:
(a) preparing a biological sample from the subject, said sample including a leukocyte;
(b) bringing a portion of the biological sample and an antibody into contact with each other, wherein the antibody is heat aggregated in the step (b);
(c) detecting expression of at least one marker of ADCC activity selected from the group consisting of tumor necrosis factor super family 15, chemokine CXCL3, and interleukin 6 in the leukocyte in (i) the portion of the sample brought into contact with the antibody and in (ii) another portion of the sample not brought into contact with the antibody by a method comprising:
(i) contacting RNA from each of the samples with a reverse transcriptase to generate complementary DNA (cDNA),
(ii) contacting said cDNA with sense and antisense primers that are specific for one of tumor necrosis factor super family 15, chemokine CXCL3, and interleukin 6 and a DNA polymerase to generate amplified DNA;
(d) comparing an expression level in portion (i) with the expression level in portion (ii); and
(e) predicting presence of the cytotoxic activity when the expression level in portion (i) is higher than the expression level in portion (ii).

2. The method according to claim 1, wherein the antibody is an FDA-approved antibody drug in a pharmaceutical composition.

3. The method according to claim 2, wherein the antibody drug is selected from the group consisting of abciximab, adalimumab, alemtuzumab, basiliximab, bevacizumab, cetuximab, daclizumab, eculizumab, efalizumab, gemtuzumab ozogamicin, ibritumomab tiuxetan, infliximab, muromonab, natalizumab, omalizumab, palivizumab, panitumumab, ranibizumab, rituximab, tositumomab, tocilizumab, golimumab, canakinumab, ustekinumab, ofatumumab, denosumab, motavizumab, raxibacumab, belimumab, ipilimumab, brentuximab vedotin, and trastuzumab.

4. The method according to claim 1, wherein the antibody is brought into contact with the biological sample with the antibody being thermally denatured or as a complex with an antigen against the antibody.

5. The method according to claim 1, wherein the detection of the expression is detection of expression of tumor necrosis factor super family 15.

6. A method for evaluating cytotoxic drug responsiveness of a patient to an antibody drug, the method comprising the steps of:
(a) preparing a biological sample from a patient, said sample including a leukocyte;
(b) bringing a portion of the biological sample and an antibody into contact with each other, wherein the antibody is heat aggregated in the step (b);
(c) detecting expression of at least one marker of cytotoxic drug responsiveness selected from the group consisting of tumor necrosis factor super family 15, chemokine CXCL3, chemokine CXCL1, and tumor necrosis factor super family 2 in the leukocyte in (i) the portion of the sample brought into contact with the antibody and in (ii) another portion of the sample not brought into contact with the antibody by a method comprising:
(i) contacting RNA from each of the samples with a reverse transcriptase to generate complementary DNA (cDNA),
(ii) contacting said cDNA with sense and antisense primers that are specific for one of tumor necrosis factor super family 15, chemokine CXCL3, chemokine CXCL1, and tumor necrosis factor super family 2 and a DNA polymerase to generate amplified DNA;
(d) comparing an expression level in portion (i) with the expression level in portion (ii); and
(e) determining that cytotoxic drug responsiveness of the patient to the antibody drug is present when the expression level in portion (i) is higher than the expression level in portion (ii).

7. The method according to claim 6, wherein the antibody drug is selected from the group consisting of an anticancer drug, an antiviral agent, an antiinflammatory agent, a rejection inhibitor, and an antitumor drug.

8. The method according to claim 7, wherein the antibody drug is trastuzumab.

9. The method according to claim 6, further comprising a step of detecting expression of a HER2 protein antigen in the patient.

10. A kit for predicting antibody-dependent cellular cytotoxic (ADCC) activity, comprising means for detecting expression of at least one marker of ADCC activity selected from the group consisting of tumor necrosis factor super family 15, chemokine CXCL3, and interleukin 6.

11. The kit according to claim 10, wherein the means for detecting the expression comprises a primer and/or a probe specific for one of tumor necrosis factor super family 15, chemokine CXCL3, and interleukin 6 and a DNA polymerase.


SEQUENCE LISTING (10 sequences)

SEQ ID NO: 1 - DNA, 23 bp, Artificial Sequence
  F-primer for TNFSF15
tgcgaagtaggtagcaactggtt

SEQ ID NO: 2 - DNA, 22 bp, Artificial Sequence
  R-primer for TNFSF15
ccattagcttgtccccttcttg

SEQ ID NO: 3 - DNA, 23 bp, Artificial Sequence
  F-primer for CXCL3
ggaattcacctcaagaacatcca

SEQ ID NO: 4 - DNA, 21 bp, Artificial Sequence
  R-primer for CXCL3
gtggctatgacttcggtttgg

SEQ ID NO: 5 - DNA, 24 bp, Artificial Sequence
  F-primer for IL-6
tcatcactggtcttttggagtttg

SEQ ID NO: 6 - DNA, 20 bp, Artificial Sequence
  R-primer for IL-6
tctgcacagctctggcttgt

SEQ ID NO: 7 - DNA, 16 bp, Artificial Sequence
  F-primer for CXCL1
ccactgcgcccaaacc

SEQ ID NO: 8 - DNA, 20 bp, Artificial Sequence
  R-primer for CXCL1
gcaggattgaggcaagcttt

SEQ ID NO: 9 - DNA, 20 bp, Artificial Sequence
  F-primer for TNFSF2
ggagaagggtgaccgactca

SEQ ID NO: 10 - DNA, 18 bp, Artificial Sequence
  R-primer for TNFSF2
tgcccagactcggcaaag